(19) United States
(12) Patent Application Publication
(10) Pub. No.: US 2009/0149512 A1
(43) Pub. Date: Jun. 11, 2009

(54) Use of Ghrelin Antagonists to the Treatment of Certain CNS Diseases

(75) Inventors: Kirsten Raun, Lyngby (DK); Lotte Bjerre Knudsen, Kalundborg (DK); Elene J.Hother Carlsen, Charlottenlund (DK); Bernd Peschke, Malov (DK); Jesper Lau, Farum (DK); Karin Rimwall, Horsholm (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/596,242

(22) PCT Filed: May 13, 2005
(86) PCT No.: PCT/EP05/52220

§ 371 Date: Sep. 18, 2007

Related U.S. Application Data
(60) Provisional application No. 60/579,578, filed on Jun. 14, 2004.

(30) Foreign Application Priority Data
May 14, 2004 (DK) PA 2004 00780

(51) Int. Cl. A61K 31/4245; A61P 25/00

(52) U.S. Cl. 514/361

(57) ABSTRACT

Ghrelin antagonists can be used for the treatment of certain CNS disorders. For example, certain oxadiazoles, preferably such being ghrelin antagonists, can be used to treat obesity, e.g., drug-induced obesity.

### FIELD OF THIS INVENTION

The present invention relates to the use of ghrelin antagonists for the treatment of certain CNS disorders, to the use of these compounds in pharmaceutical compositions, to pharmaceutical compositions comprising the compounds, and to methods of treatment employing these compounds or compositions. These compounds can be used to treat obesity, e.g. drug-induced obesity.

### BACKGROUND OF THIS INVENTION

Ghrelin was originally discovered as an endogenous peptide that stimulates growth hormone (GH) secretion. Ghrelin is now recognized as being an orexigenic hormone of major importance, and ghrelin antagonists are under development as anti-obesity agents (Carpino in Expert Opinion Ther. Patents 2002, 12 (11), 1599-1618; Guillano in FEBS 2003, 552, 105-109; Ukkola in Eur. J. Int. Med. 2003, 14, 351-356; and Kojima in Curr. Opi. Pharm. 2002, 2, 665-668). Recently, ghrelin has also been described as having central nervous system (CNS) effects giving rise to increased anxiety-like behaviour and increased memory retention in rats (Carlini et al. in Biochem. Biophys. Res. Comm. 2002, 299, 739-743; and Carlini et al. in Biochem. Biophys. Res. Comm. 2004, 313, 635-641).

Disorders associated with decreased wakefulness and/or decreased attention cause severe medical problems. Such disorders include narcolepsy (Dauvilliers in Clin.-Neurophysiol. 2003, 114 (11), 2000-2017), sleep-wake disturbances (disturbances of the sleep-wake cycle), daytime “sleepiness” in subjects suffering from obstructive sleep apnoea and similar disorders (J. Sleep Res. 2002, 11, 1-16), Attention Deficit and Hyperactivity Disorder (ADHD) (Maidment in Ann. Pharmacoth. 2003, 37(12), 1884-1890), Alzheimer's disease (Corey-Boom in Int. Psyochoger. 2002, 14, 51-75), multiple sclerosis (Bobholz in Curr. Opi. Neurol. 2003, 16 (3), 283-288), Parkinson's disease, dementia of non-Alzheimer origin (for example, vascular dementia) and defective short- and long-term memory, and they may also include depression and schizophrenia (Garcia-Toro in Prog. Neur. Psychopharm. Biol. Psych. 2003, 27 (1), 37-42; Elvevag in Cit. Rev. Neurobiol. 2000, 14 (1), 1-21).

Examples of drugs which frequently result in body-weight gain in human subjects include the so-called “atypical antipsychotics”. The atypical antipsychotics are a group of antipsychotics (neuroleptic drugs) that have been introduced in the last decade or so, and which include, for example, sulpiride (for example, Dolmatil™), amisulpiride (for example, Solian™), clozapine (for example, Clozaril™), risperidone (for example, Risperdal™), olanzapine (for example, Zyprexa™), quetiapine (for example, Seroquel™), ziprasidone (for example, Geodon™) and aripiprazole (for example, Abilify™). The tendency toward weight-gain appears to be especially severe in connection with, in particular, treatment with clozapine and olanzapine.

The atypical antipsychotics—which are increasingly used to treat severe psychiatric disorders such as schizophrenia, schizotypal disorders, schizoaffective disorders, affective disorders, delusional disorders and psychosis caused by use of psychoactive substances—are less likely to cause extrapyramidal side-effects than the earlier-generation, so-called “typical antipsychotics” (conventional antipsychotics) [examples of which are chlorpromazine (for example, Thorazine™), perphenazine (for example, Trilafon™), trifluoperazine (for example, Stelazine™), thiothixene (for example, Navane™), haloperidol (for example, Haldol™), fluphenazine (for example, Prolixin™) and thioridazine (for example, Mellaril™)], and may in addition be more effective than the latter in relieving associated symptoms such as withdrawal, cognitive problems and lack of energy. Nevertheless, the well-known tendency of atypical psychotics to cause body-weight gain or obesity is a major problem with respect to achieving patient compliance.

Other psycho-active drugs which are known to be capable of causing body-weight gain include: typical antipsychotics (vide supra); tri- and tetracyclic antidepressants [such as mirtazapine (for example, Remeron™)]; antimania lithium drugs (such as lithium carbonate); and the anti-epilepsy drug valproat (for example, Deprakine™ Retard or Leptilan™).

Other classes of drugs which are also known to be capable of causing body-weight gain include: certain steroids; tamoxiphen; certain antidepressive drugs (other than tri- and tetracyclic antidepressants); and certain immunosuppressive drugs.

The underlying cause of body-weight gain or development of obesity in association with drug treatments of the kind outlined above have in general not been clearly identified, but an increase in appetite and/or a decrease in metabolic rate often appears to be involved. There are currently very few approved drugs on the market for controlling appetite or achieving an increase in metabolic rate. One such drug, viz. sibutramine (available, for example, as Meridia™), which reduces appetite and is able to increase metabolic rate to some extent, acts on the CNS by modulating the levels of serotonin neurotransmitters, and is generally regarded as being contraindicated for treatment of patients with current or previous psychiatric disorders.

One object of this invention, among others, is to overcome or ameliorate at lest some of the disadvantages of the prior art. Hence, not all the objects mentioned below may be fully overcome or ameliorated.

One object of this invention is to furnish compounds which, effectively, can be used to treat or prevent certain CNS diseases.

Another object of this invention is to furnish compounds which, effectively, can be used to treat or prevent obesity.

Another object of this invention is to furnish compounds which, effectively, can be used to treat or prevent drug-induced obesity.

These and other aspects, features, objects, and advantages of this invention (including numerous particular aspects of the invention) will be apparent to those of ordinary skill in the art from the description of this invention provided herein.

### DEFINITIONS

The terms “ghrelin antagonist”, “substance which is a ghrelin antagonist”, “ghrelin-antagonistic substance” and “ghrelin-antagonistic compound” and similar expressions, in the context of this invention, unless otherwise stated or clearly contradicted by context, refer to a substance that acts on ghrelin per se or on the ghrelin receptor(s) (vide supra) so as to cause an inhibition of binding of ghrelin to the receptor(s). In general, substances of interest include substances that bind to ghrelin receptor(s) so as to cause a functional antagonism of the physiological response produced by binding of ghrelin to the receptor(s). In a preferred embodiment of this invention, ghrelin antagonists are compounds exhibiting an IC₅₀ value of less than 10 μM when tested by the test below for measurement of binding to human GHS-R1a. Furthermore, one has to make the test below for determination of agonism or antagonism.

A review of the general area of ghrelin and its binding to the relevant receptor or receptors has been published (C. Y. Bowers in J. Clin. Endocrinol. Metab. 2001, 86 (4), 1464-1469). The relevant receptor or receptors has/have often been broadly designated “Growth Hormone Secretagogue Receptor” (herein abbreviated to GHS-R), although it now appears that the primary ghrelin receptor—at least in mammals, including humans—is a particular sub-type of GHS-R often referred to as GHS-R1a. It is, however, possible that binding to other receptor sub-types, such as that often referred to as GHS-R1b, may be relevant in relation to at least some of the effects exerted by ghrelin on the organism.

The term obesity implies an excess of adipose tissue. In this context obesity is best viewed as any degree of excess adiposity that imparts a health risk. The cut off between normal and obese individuals can only be approximated, and the health risk imparted by the obesity is probably a continuum with increasing adiposity. In the context of the present invention, individuals with a body mass index (BMI=body weight in kilograms divided by the square of the height in meters) above 25 are to be regarded as obese. In one embodiment in the context of this invention, a human subject is said to be obese when the subject has a BMI of 30 or more.

Treatment of obesity, e.g. drug-induced obesity, in this document includes any lowering of body weight or decrease in body fat in a subject, including but not limited to those suffering from conditions described as overweight or obesity.

Obesity also covers treatment of Binge eating disorder (BED), a fairly new diagnosable disorder—see, for example, Int. J. Obesity, 2002, 26, 299-307, and Curr. Opin.-Pshyciatry, 17, 43-48, 2004. BED is characterised by binge eating episodes as is bulimia nervosa (BN). However, subjects with BED do not, contrary to patients with bulimia nervosa, engage in compensatory behaviours, such as, for example, self-induced vomiting, excessive exercise, and misuse of laxatives, diuretics or enemas. Studies have shown that 1-3% of the general population suffer from BED, whereas a higher prevalence (up to 25-30%) have been reported for obese patients [Int. J. Obesity, 2002, 26, 299-307]. These numbers show that BED subjects may or may not be obese, and that obese patients may or may not have BED, i.e., that the cause of the obesity is BED. However, a proportion of subjects with BED eventually become obese due to the excess calorie intake. Laboratory studies have shown that BED patients consumed more dessert and snack (rich in fat and poor in proteins) than did an obese control group [Int. J. Obesity, 2002, 26, 299-307], and the compounds and methods of the present invention are particular well-suited for treatment of BED.

BN is characterised by the same binge eating episodes as is BED, however, BN is also characterised by the above mentioned compensatory behaviour. A proportion of subjects with BN will eventually become obese to the extent that the compensatory behaviour cannot fully compensate the excess calorie intake. Studies have compared binges of patients with BN and with BED concluding that binges in subjects with BN were higher in carbohydrates and sugar content than those of subjects with BED. No difference was, however, found in the number of consumed calories [Int. J. Obesity, 2002, 26, 299-307]. The compounds and methods of the present invention are particular well-suited for the treatment of BN.

Craving for food or the intense desire to eat a particular food is normally associated with energy dense food, such as fatty or carbohydrate-rich food [Appetite, 17, 177-185, 1991; Appetite, 17, 167-175, 1991]. Examples of such foods include chocolate, biscuits, cakes and snacks. A proportion of food cravers eventually become obese due to the excess calorie intake. The compounds and methods of the present invention are particular well-suited for the treatment of food craving, in particular craving for fatty or carbohydrate-rich food.

A snack is typically a light, casual, hurried convenience meal eaten between real meals. Snacks are typically fatty and carbohydrate-rich. Studies have shown that there is an increasing prevalence of snacking, especially among US children, and that snacking is a significant cause for the increase in BMI in, for example, children [J. Pediatrics, 138, 493-498, 2001; and Obes. Res., 11, 143-151, 2003]. A shift towards more healthy snacks could probably arrest or change the increase in BMI which has taken place over the last years. Medicaments which are capable of shifting food preferences from fatty and carbohydrate-rich food to low-fat glycemic index low food are desired. The compounds and methods of this invention are useful in diminishing the amount of snacking or in changing the preference of snack to healthier snack.

Several drugs, particularly antipsychotics and certain steroids, are known to induce severe weight gain. A weight gain of about 7% over ideal body weight is considered a significant health risk due to the accompanying obesity that might lead to diseases such as diabetes and cardiovascular diseases as well as a multitude of other obesity related diseases including cancer. With the average weight and BMI rapidly increasing over the whole world, the problem becomes even more severe.

The so-called atypical antipsychotic drugs are increasingly used to treat severe psychiatric diseases, among those, schizophrenia, schizotypal disorders, schizoaffective disorders, affective disorders, delusional disorders, and psychosis caused by use of psychoactive substances. Atypical anti psychotics include amisul pride, sulpi ride, clozapine, risperidone, olanzapine, quetiapine, ziprasidone, and aripiprazole. Typical antipsychotics include chlorpromazine, perpherazine, thifluoperazine, thiothixene, haloperidol, and fluphenzine. Atypical antipsychotics are less likely to cause extrapyrimidal side effects than the typical antipsychotics. In addition, atypical antipsychotics work on the negative symptoms and cognitive disturbances as well, which the typical antipsychotics generally do not.

Among the side effects of the atypical antipsychotics is weight gain, which in some cases is very pronounced. Clozapine and olanzapine are, especially, known to cause severe weight gain. The weight gain is an important side effect since it lowers patient compliance. Furthermore, patients with weight gain are at increased risk to develop diabetes, and a weight gain in this population would probably lead to even more cases of diabetes compared with the background population. Also, the typical antipsychotics and other CNS-active drugs such as lithium, mirtazapine, tri- and tetracyclic antidepressants, and valproat can cause weight gain.

### SUMMARY OF THIS INVENTION

Important aspects of this invention relate to: a method for treatment of a disorder associated with decreased wakefulness, decreased cognition, decreased memory capacity or decreased attention, the method comprising administering to a subject in need thereof an effective amount of a substance which is a ghrelin antagonist; and to the use of such a ghrelin antagonist in the manufacture of a medicament for treatment of such a disorder.

Further aspects of this invention relate to, inter alia, a method for treatment or prophylaxis of obesity, e.g. drug-induced obesity, the method comprising administering to a subject in need thereof an effective amount of a substance which is a ghrelin antagonist; and to the use of such a ghrelin antagonist in the manufacture of a medicament for treatment of obesity, e.g. drug-induced obesity.

Still further aspects of this invention include the treatment of obesity, e.g. drug-induced obesity, by treatment with certain ghrelin-antagonistic substances.

### DETAILED DESCRIPTION OF THIS INVENTION

In one aspect, the invention described herein provides methods of modulating CNS-related disorders by the administration of ghrelin antagonists. The inventors have now surprisingly found that ghrelin antagonists have other important CNS effects than those already known, in that they are able, inter alia, to produce increased wakefulness and attention. In a particularly advantageous aspect, as already indicated to some extent above, it has now, surprisingly, been found that wakefulness and attention in a subject may be increased by administering to the subject a ghrelin antagonist, i.e. a substance which acts as an antagonist with respect to binding of ghrelin to the ghrelin receptor.

It thus appears that ghrelin antagonists can be used to treat, inter alia, disorders among those listed above, i.e. disorders such as narcolepsy, sleep-wake disturbances, daytime sleepiness or drowsiness in subjects suffering from obstructive sleep apnoea (or from other conditions causing daytime sleepiness or drowsiness), ADHD, Alzheimer's disease, Parkinson's disease, non-Alzheimer dementia, depression and schizophrenia. Such treatment can reduce the effects or symptoms of any of such conditions, ameliorate the state of such conditions, etc.

The administration of ghrelin antagonists may also or alternatively be used generally to promote wakefulness/alertness in a subject in need of increased alertness having or lacking such conditions (such as a subject recognized as lacking alertness due to current physiological conditions, such as fatigue, performing a task that required maintained alertness, or suffering from a condition such as excessive daytime sleepiness (EDS)). Alertness in humans, for example, can be measured by any number of suitable known assays and indicators including, e.g., objective tests such as the Multiple Sleep Latency Test (MSLT) and the Maintenance of Wakefulness Test (MWT), subjective standards such as the Epworth sleepiness scale (ESS), or physiological tests such as electro-encephalography (EEG) and polysomnographic studies.

The method of this invention may in principle be practiced using any ghrelin-antagonistic substance, unless otherwise indicated. Examples of known ghrelin-antagonistic substances (GHS-R1a antagonists) that may be employed in the context of this invention are believed to include the following: the peptidic antagonist denoted L-756867 (i.e. D-ArgPro-Lys-Pro-D-Phe-Gln-D-Trp-Phe-D-Trp-Leu-Leu-NH₂ [attached as SEQ ID1]); a “substance P” derivative; (D-Lys3)-GHRP-6 (i.e. His-D-Trp-D-Lys-Trp-D-Phe-Lys-NH₂ [attached as SEQ ID2]); and a non-peptidyl antagonist denoted L-692400 [see P. A. Carpino in Expert Opin. Ther. Patents 2002: 12 (11): 1599-1618, and references cited therein]. Ghrelin-antagonistic substances described or disclosed in WO 01/87335, in WO 01/92292, in WO 02/08250, in WO 2003/004518, in WO 2004/004772 and in WO 2004/013274 may likewise be of relevance in the context of this invention.

Examples of other ghrelin antagonists are the following: cyclo(-His-D-Trp-Ala-Trp-D-Phe-) with the formula:

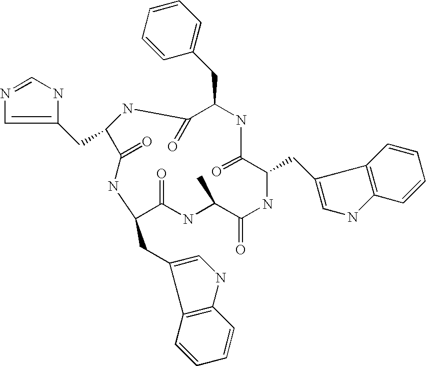

(2E)-4-(1-aminocyclobutyl)but-2-enoic acid N-((1R)-1-diphenethylcarbamoyl-2-(2-naphthyl)ethyl)-N-methylamide with the formula:

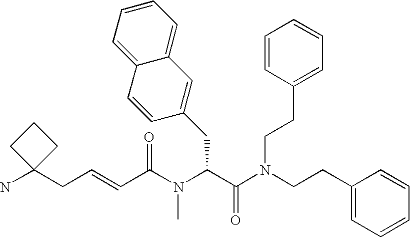

(E)-5-amino-5-methylhex-2-enoic [(R)-2-(1-(benzofuran-7-yl)-7-chloro-8-methoxy-1,2,4,5-tetrahydrobenzo[d]azepin-3-yl)-1-(benzyloxymethyl)-2-oxoethyl]amide with the formula:

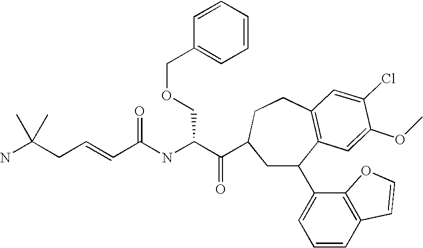

2-amino-N-[(1R)-1-{N-[(1R)-1-(N′,N′-dimethylhydrazinocarbonyl)-3-phenylpropyl]-N-methylcarbamoyl}-2-(1H-indol-3-yl)ethyl]-2-methylpropionamide with the formula:

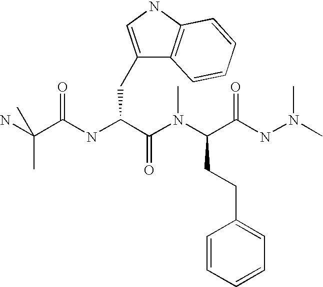

and 2-[(1R)-1-((2E)-5-amino-5-methylhex-2-enoylamino)-2-(2-naphthyl)ethyl]-5-phenyloxazole-4-carboxylic acid methyl ester with the formula:

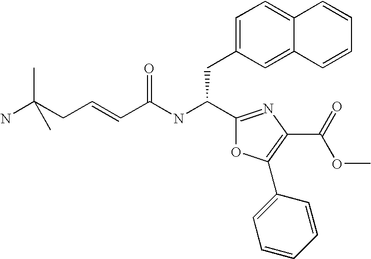

In current drug treatments for some disorders of the type in question, the drugs themselves can lead to body-weight gain or obesity in various degrees of severity. A body-weight gain of about 7% or more in a human subject compared to ideal body weight is considered to constitute a significant health risk owing to the attendant increased risk of development of, for example, diabetes, cardiovascular disease and other obesity-related diseases and disorders (such as various forms of cancer), and with the current global trend towards increasing average body weight, and thereby increasing the body mass index (BMI), the problem becomes even more severe.

In relation to the above, an aspect of this invention provides a method for treatment or prophylaxis [i.e. a method for achieving a detectable decrease of, delay in the onset or severity of, circumvention and/or prevention in a particular subject and/or in a significant proportion of subjects in a population of similar subjects, e.g., at least about 25%, at least about 33%, at least about 50%, at least about 75% or more (such as about 30-100%), as may be determined by, for example, clinical human or veterinary trials] of obesity, e.g. drug-induced body-weight gain or obesity, comprising administering to a subject in need thereof an effective amount of a ghrelin antagonist. The drug in question may, for example, be any one of those types or examples mentioned above as having tendency to cause body-weight gain or obesity in a subject. In a further aspect, the subject in question has undergone, and/or is undergoing, treatment with a drug—such as an atypical antipsychotic—for the treatment of a disorder associated with decreased wakefulness, decreased cognition, decreased memory capacity or decreased attention.

In yet another, closely related, aspect of this invention, a ghrelin antagonist may be administered to a subject that for any reason has undergone, and/or is undergoing, treatment with an atypical antipsychotic (i.e. irrespective of the disease, condition or disorder which is the justification for treatment with an atypical antipsychotic) in order to decrease, circumvent or prevent body weight increase in the subject.

In a still further aspect of this invention, a ghrelin antagonist may be administered to a subject in order to inhibit intake (ingestion) of high-fat and/or high-carbohydrate food by the subject. Treatment of a subject with a ghrelin antagonist causes a change in food preference, leading to a relatively reduced intake of high-fat and/or high-carbohydrate food (particularly high-carbohydrate food with a high mono- and/or di-saccharide content), i.e. to a relatively reduced intake of energy (calories) originating from fats and/or carbohydrates.

Still further aspects of this invention relate to the use of a ghrelin antagonist in the manufacture of a medicament for the treatment of any of the disorders, diseases or conditions mentioned above as being treatable in the context of other aspects of this invention.

It will be apparent that the various aspects of this invention are applicable not only to human subjects, but may also be of value in relation to treatment of animals, particularly mammals.

Other ghrelin antagonists of value in the context of this invention include ghrelin-antagonistic compounds within the scope of the following general Formula I:

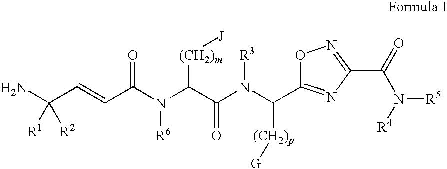

wherein R¹ and R² independently of each other are hydrogen or C₁₋₆alkyl, or R¹ and R² taken together form a C₂₋₅alkylene group; J is a group

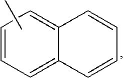

optionally substituted with one or more C₁₋₆alkyl or halogen; m is 1, 2 or 3; R³ is C₁₋₆alkyl; p is 1, 2 or 3; G is a group

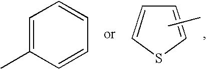

optionally substituted with one or more C₁₋₆alkyl or halogen; R⁴ and R⁵ independently of each other are hydrogen or C₁₋₆alkyl; and R⁶ is hydrogen or C₁₋₆alkyl, preferably hydrogen; and pharmaceutically acceptable salts thereof.

Compounds of Formula I can have one or more asymmetric centres, and any and all optical isomers in the form of separated, pure or partially purified optical isomers or racemic mixtures thereof are included within the scope of Formula I. Both E and Z geometric isomers (with respect to the olefinic double bond to the left in the structure of Formula I as depicted above) are likewise included within the scope of Formula I.

According to one embodiment of this invention, R¹ and R² are both alkyl, preferably methyl.

According to another embodiment of this invention, J is 2-naphthyl.

According to another embodiment of this invention, m is one.

According to another embodiment of this invention, R³ is methyl.

According to another embodiment of this invention, p is one.

According to another embodiment of this invention, G is phenyl.

According to another embodiment of this invention, R⁴ is methyl.

According to another embodiment of this invention, R⁵ is hydrogen or methyl.

According to another embodiment of this invention, R⁶ is hydrogen or methyl. In the context of Formula I, the term “C₁₋₆alkyl” is intended to include straight-chain (linear), branched and cyclic alkyl groups of from 1 to 6 carbon atoms. Relevant linear C₁₋₆alkyl groups are methyl, ethyl, propyl, butyl, pentyl and hexyl. Examples of branched C₁₋₆alkyl groups are isopropyl, sec-butyl, tert-butyl, isopentyl and isohexyl. Examples of cyclic groups (C₃₋₆cycloalkyl groups) are cyclopropyl, cyclobutyl, cyclopentyl and cyclohexyl. The term “C₁₋₆alkyl” in the present context likewise includes, for example, cycloalkyl-substituted alkyl groups having from 1 to 6 carbon atoms, examples of which include groups such as (cyclopropyl)methyl, (cyclopropyl)ethyl, (cyclopropyl)propyl, (cyclobutyl)methyl, (cyclobutyl)ethyl and (cyclopentyl)methyl. Particularly suitable C₁₋₆alkyl groups are often chosen among C₁₋₃alkyl groups, i.e. methyl, ethyl, propyl, isopropyl and cyclopropyl.

The term “C₂₋₅alkylene group” (i.e. C₂₋₅alkandiyl group) is intended to include both straight-chain (linear) and branched alkandiyl groups of from 2 to 5 carbon atoms. Relevant linear groups are: —CH₂—CH₂—; —CH₂—CH₂—CH₂—; —CH₂—(CH₂)₂—CH₂—; and —CH₂—(CH₂)₃—CH₂—. Examples of suitable branched groups include: —CH₂—CH(CH₃)—; —CH₂—CH(CH₃)—CH₂—; —CH₂—CH₂—CH(CH₃)—; —CH₂—(CH₂)₂—CH(CH₃)—; and —CH₂—CH₂—CH(CH₃)—CH₂—.

The term “halogen” includes Cl, F, Br and I. Particularly suitable halogens in the context of this invention are Cl and F.

An example of a ghrelin antagonist within the scope of Formula I, above, is a diastereoisomer of the following compound: (2E)-4-Amino-4-methylpent-2-enoic acid {(R)-1-[N-[1-(3-(N-methylcarbamoyl)-1,2,4-oxadiazol-5-yl)-2-phenylethyl]-N-methylcarbamoyl]-2-(2-naphthyl)ethyl} amide:

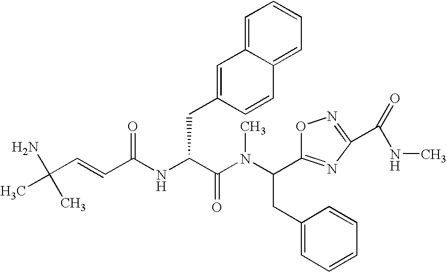

more specifically the diastereoisomer referred to in the present specification with claims as “Diastereoisomer 2” or “D2”, which exhibits the following ¹H-NMR spectroscopic data (in DMSO-d₆, as the acetate salt of Diastereoisomer 2): δ=1.30 (s, 3H); 1.32 (s, 3H); 1.95 (s, 3H); 2.55 (d, 2H); 2.80 (d, 3H); 3.00 (s, 3H); 3.30 (dd, 1H); 3.50 (dd, 1H); 5.00 (q, 1H); 6.05 (dd, 1H); 6.10 (d, 1H); 6.60 (d, 1H); 7.15-7.90 (m, 12H); 8.70 (d, 1H); and 8.95 (q, 1H); and which is the more slowly eluting diastereoisomer in HPLC separation of the two diastereoisomers (Diastereoisomer 1 and Diastereoisomer 2) of this compound performed as described on pages 52-53 of WO 96/22997.

Examples of other compounds of Formula I are (2E)-4-amino-4-methylpent-2-enoic acid {1-[N-[1-(3-(N,N-dimethylcarbamoyl)-1,2,4-oxadiazole-5-yl)-2-phenylethyl]-N-methylcarbamoyl]-2-(2-naphthyl)ethyl} amide (diastereomer 2) with the formula:

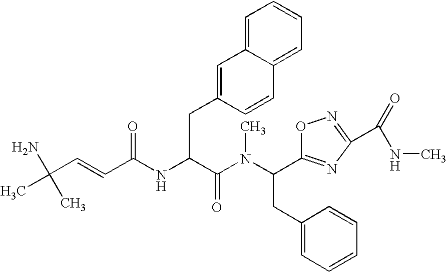

and (2E)-4-amino-4-methylpent-2-enoic acid N-{(R)-1-[N-[1-(3-(N,N-dimethylcarbamoyl)-1,2,4-oxadiazole-5-yl)-2-phenylethyl]-N-methylcarbamoyl]-2-(2-naphthyl)-ethyl}-N-methylamide with the formula:

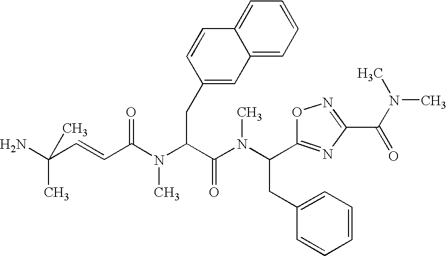

A method of general applicability in the preparation of compounds of Formula I is “General Method E” described on pages 24-25 of WO 96/22997, and a person of ordinary skill in the art will be able on the basis thereof to prepare desired compounds within the scope of Formula I.

A further aspect of this invention relates to ghrelin-antagonistic compounds within the scope of Formula I as defined above, including the specific compound disclosed and described above, and pharmaceutically acceptable salts thereof.

A still further aspect of this invention relates to a pharmaceutical composition comprising, as an active ingredient, a ghrelin-antagonistic compound of Formula I, or a pharmaceutically acceptable salt thereof, as disclosed herein together with a pharmaceutically acceptable carrier or diluent.

### Pharmaceutically Acceptable Salts

As already indicated, pharmaceutically acceptable salts of ghrelin-antagonistic substances are included, in addition to the substances per se, in the context of this invention. Such salts include, in general, pharmaceutically acceptable acid addition salts, pharmaceutically acceptable metal salts, ammonium salts and alkylated ammonium salts. Acid addition salts include salts of inorganic acids as well as organic acids. Representative examples of suitable inorganic acids include hydrochloric, hydrobromic, hydriodic, phosphoric, sulfuric, sulfamic and nitric acids. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, ethylenediaminetetraacetic (EDTA), p-aminobenzoic, glutamic, benzenesulfonic and p-toluenesulfonic acids. Further examples of pharmaceutically acceptable inorganic or organic acid addition salts include the pharmaceutically acceptable salts listed in J. Pharm. Sci. 1977, 66, 2. Examples of metal salts include lithium, sodium, potassium, calcium and magnesium salts. Examples of ammonium and alkylated ammonium salts include ammonium, methylammonium, dimethylammonium, trimethylammonium, ethylammonium, hydroxyethylammonium, diethylammonium, butylammonium and tetramethylammonium salts.

Acid addition salts are of particular relevance in relation to compounds of Formula I as disclosed herein.

Also included in the context of this invention are hydrated forms (hydrates) of ghrelin antagonists or of pharmaceutically acceptable salts thereof.

### Pharmaceutical Formulation/Compositions

The compounds of this invention may be administered alone or in combination with pharmaceutically acceptable carriers or excipients, in either single or multiple doses. The pharmaceutical compositions according to this invention may be formulated with pharmaceutically acceptable carriers or diluents as well as any other known adjuvants and excipients in accordance with conventional techniques, such as those disclosed in Remington in The Science and Practice of Pharmacy, 19^{th} Edition, Gennaro, Ed., Mack Publishing Co., Easton, Pa. (1995).

The pharmaceutical compositions may be specifically formulated for administration by any suitable route such as the oral, rectal, nasal, pulmonary, topical (including buccal and sublingual), transdermal, intracisternal, intraperitoneal, vaginal or parenteral (including subcutaneous, intramuscular, intrathecal, intravenous and intradermal) route. It will be appreciated that the choice of route will depend on the general condition and age of the subject to be treated, the nature of the condition to be treated and the nature of the active ingredient (ghrelin antagonist) in question.

Pharmaceutical compositions for oral administration include solid dosage forms such as capsules, tablets, dragees, pills, lozenges, powders and granules. Where appropriate, they can be prepared with coatings, such as enteric coatings, or they can be formulated so as to provide controlled release of the active ingredient, such as sustained or prolonged release, according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use. Depot injectable formulations are also contemplated as being within the scope of this invention.

Other possible administration forms include suppositories, sprays, ointments, cremes, gels, inhalants, dermal patches and implants.

A typical oral dosage of a compound employed according to this invention will be in the range of from about 0.0001 to about 100 mg/kg body weight per day, often from about 0.001 to about 50 mg/kg body weight per day, such as from about 0.01 to about 25 mg/kg body weight per day, administered in one or more doses per day, such as 1-3 doses per day.

The formulations or compositions may conveniently be presented in unit dosage form in accordance with methodology well known to those skilled in the art. A typical unit dosage form for oral administration one or more times per day, such as 1-3 times per day, may contain from about 0.05 to about 2000 mg, often from about 0.1 to about 500 mg, such as from about 0.5 mg to about 200 mg of a compound employed according to this invention.

For parenteral routes, such as intravenous, intrathecal, intramuscular and similar routes of administration, doses will typically be of the order of about half the dose employed for oral administration.

The compounds of Formula I disclosed herein will generally be utilized as the free substance or as a pharmaceutically acceptable salt thereof, notably as an acid addition salt thereof. Compounds of Formula I contain a free base (amino) functionality, and such salts are suitably prepared in a conventional manner by treating a solution or suspension of the free base form of the compound with, typically, one equivalent (chemical equivalent, i.e. acid-base equivalent) of a pharmaceutically acceptable acid, for example an inorganic or organic acid chosen among the representative examples thereof mentioned above.

For parenteral administration, solutions of the present compounds in sterile aqueous solution, aqueous propylene glycol or sesame or peanut oil may be employed. Such aqueous solutions should be suitably buffered if necessary, and the liquid diluent first rendered isotonic using sufficient saline, glucose, mannitol or other pharmaceutically acceptable tonicity-adjusting substance. The aqueous solutions are particularly suitable for intravenous, intramuscular, subcutaneous and intraperitoneal administration. The sterile aqueous media employed are all readily available in accordance with standard methodology well known to persons of ordinary skill in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylenes and water. Similarly, the carrier or diluent may include a sustained-release material known in the art, such as glyceryl monostearate or glyceryl distearate, alone or mixed with a wax. The pharmaceutical compositions formed by combining the compounds of this invention and the pharmaceutically acceptable carriers are then readily administered in a variety of dosage forms suitable for the disclosed routes of administration. The formulations may conveniently be presented in unit dosage form by methods well known in the art of pharmacy.

Formulations of this invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form, or it can be in the form of a troche or lozenge. The amount of solid carrier will vary widely, but for human administration will usually be from about 25 mg to about 1 g. If a liquid carrier is used, the preparation may be in the form of a syrup, emulsion, soft gelatine capsule or sterile injectable liquid, such as an aqueous or non-aqueous liquid suspension or solution.

As already indicated, ghrelin antagonists as employed in the context of this invention may be administered not only to humans, but also to animals—particularly mammals—in need thereof. Such mammals may include both domesticated animals, for example household pets or farm animals, and non-domesticated animals.

If appropriate, a pharmaceutical composition of this invention may comprise a ghrelin antagonist of this invention in combination with one or more other pharmacologically active substances.

The invention also provides a method of promoting the use and/or sale of a composition comprising one or more ghrelin antagonists, comprising distributing information about the usefulness of ghrelin antagonists in any of the therapeutic and/or prophylactic methods of the invention (e.g., promoting alertness in a subject), such as through television, radio, or internet advertising; mass mailing and mass e-mailing; telemarketing; funding, hosting, or holding scientific meetings, lectures, presentations, etc., regarding such methods with patients, business professionals, policy makers and/or health care providers; fielding or licensing a staff of salespeople, medical/scientific liaisons and the like so as to educate pharmacists, doctors, nurses and other health care providers about such methods; distributing media at meetings relating to such conditions or disorders; awarding grants or other funds related to published research advocating the practice of such methods; providing information to key opinion leaders in the treatment of such disorders and conditions; and the like, so as to promote the use and/or sale of such compositions.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents. The mentioning herein of references is no admission that they constitute prior art.

Herein, the word “comprise” is to be interpreted broadly meaning “include”, “contain” or “comprehend” (EPO guidelines C 4.13).

This invention includes all modifications and equivalents of the subject matter recited in the claims appended hereto as permitted by applicable law.

The following examples and tests are offered by way of illustration, not by limitation.

### EXPERIMENTAL SECTION

### Measurement of Binding to Human GHS-R1a

Isolated crude membranes from BHK cells stably expressing the human receptor GHS-R1a (see, for example, Hansen et al. in Eur. J. Endocrinol. 141, 180-189) are suspended in homogenising buffer (2.5 mM Tris-base, 2.5 mM EDTA, 10 mM MgCl₂ and 30 μg/ml bacitracin) to a concentration of 0.5 mg protein/ml and added to microtiter plates to a final total concentration of 5 μg protein/ml together with ¹²⁵I ghrelin (20,000 cpm/well) and with or without different concentrations (0.01 nM-10 μM) of test compound and binding buffer (2.5 mM Tris-base, 2.5 mM EDTA, 10 mM MgCl₂ and 0.5% BSA) up to a total volume of 250 μl. Non-specific binding is obtained by adding 10 μM ghrelin. The membranes are incubated at 30° C. for 60 minutes, and bound radioligand is separated from free radioligand by washing with binding buffer through GF/B filters (Whatman, Kent, UK) pre-treated with 0.5% polyethylenimine for 60 minutes. The radioactivity on the filters is counted in Optiphase™ ‘HiSafe 3’ (Wallac, Turku, Finland).

In the calculation, specific binding is defined as the difference between total binding and non-specific binding, defined as ¹²⁵I ghrelin binding in the absence and presence of 10 μM unlabeled ghrelin, respectively. Displacement curves are constructed using non-linear regression. Using the maximal inhibition (Eₘₐₓ) value determined thereby for the test compound in question, the potency (IC₅₀) is calculated as the dose inducing half-maximal inhibition.

Compounds exhibiting an IC₅₀ value less than 10 μM are considered to be ghrelin agonists or antagonists.

### Determination of Agonism or Antagonism

In order to distinguish between ghrelin agonism and ghrelin antagonism for a test compound determined, as described above, to be either an agonist or antagonist with respect to ghrelin, the Ca²⁺ release assay described on page 12 of WO 01/56592 may be employed.

### Assessment of Level of Wakefulness/Activity

The effect of an administered substance (for example a ghrelin antagonist) on the level of wakefulness or activity in animal subjects (for example, mice) may be assessed by means of an activity test in which the primary measurement parameter is locomotor activity.

Baseline activity is determined for all the animals 1-2 days prior to administration of saline (placebo) or ghrelin antagonist. The animals are subsequently monitored, either acutely or at the end of a more sub-chronic or chronic treatment lasting several days or weeks, for behavioural sensitization. On testing days, the animals are allowed to acclimatize to the behavioural monitors for 30 minutes prior to data accumulation if the monitors in question are different from their “home” cages. Activity is recorded for at least 1 hour on each of the testing days (baseline and post-treatment test). In a suitable arrangement, transparent chambers of Plexiglas™ are equipped with aluminium frames on which are mounted infrared light emitters and detectors. The detectors are interfaced to a personal computer (PC). Behavioural or psychomotor activity is defined as the total number of light-beam interruptions recorded during each testing session. A test compound is defined as active primarily on the basis of an increase in the rodent's horizontal locomotor response (horizontal activity), although increased locomotor response also involves increased stereotypic and rearing behaviours.

### Assessment of Level of Attention

Level of attention in animal subjects (for example, mice) may be assessed by behavioural testing in which the measurement parameter is the time taken to solve a problem requiring attentiveness. A suitable setup is the so-called “Morris water maze” test, in which a test animal, placed in different starting positions, must find the location of a submerged platform.

Other types of tests assessing spatial learning could be performed to assess level of attention. An example hereof could involve the use of a floor surface that gives rise, sequentially, to emission of intense light or sound when touched by test animals. The animals have to locate and touch a specific area on the floor in order to turn off the stimuli.

### Example 1

The ghrelin antagonist D2 (vide supra; prepared, for example, according to “General Method E” described on pages 24-25 of WO 96/22997) was administered intraperitoneally (abbreviated as i.p.) in saline solution once daily in a dose of 20 mg/kg body weight to diabetic ob/ob mice (male, 8 months old) for 14 days. Control animals received saline. The ob/ob mice exhibit a significantly decreased level of locomotor activity compared to normal mice. They are lethargic and sleep a lot. It was observed that animals treated with the ghrelin antagonist D2 became surprisingly active and awake for the duration of the study, as assessed using the light-beam interruption method described above. When observed, the treated animals were awake and interested in their environment. They also became increasingly difficult to catch.

1. A method for treatment of a decrease in wakefulness, cognition, memory capacity, attention, or a combination of any thereof, comprising administering to a subject in need thereof an effective amount of a ghrelin antagonist.

2. The method according to claim 1 wherein the subject suffers from narcolepsy, sleep-wake disturbances, daytime sleepiness or drowsiness associated with obstructive sleep apnoea, ADHD, Alzheimer's disease, non-Alzheimer dementia, Parkinson's disease, depression, or schizophrenia.

3. A method for treatment or prophylaxis of drug-induced body-weight gain or obesity comprising administering to a subject in need thereof an effective amount of a ghrelin antagonist.

4. The method according to claim 3, wherein the drug-induced body-weight gain or obesity is a consequence of treatment of said subject with an atypical antipsychotic.

5. A method for inhibiting intake of high-fat, high-carbohydrate food comprising administering to a subject in need thereof an effective amount of a ghrelin antagonist.

6. (canceled)

7. The method according to claim 1, wherein the ghrelin antagonist consists essentially of a compound within the general Formula I: 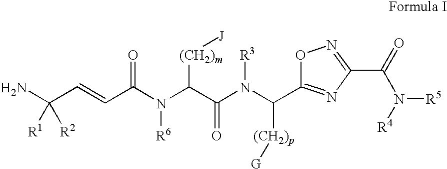
wherein R¹ and R² independently of each other are hydrogen or C₁₋₆alkyl, or R¹ and R² taken together form a C₂₋₅alkylene group; J is a group of the formula 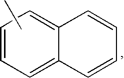
optionally substituted with one or more C₁₋₆alkyl or halogen; m is 1, 2 or 3; R³ is C₁₋₆alkyl; p is 1, 2 or 3; G is a group of the formula 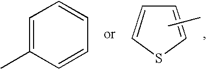
optionally substituted with one or more C₁₋₆alkyl or halogen; R⁴ and R⁵ independently of each other are hydrogen or C₁₋₆alkyl; and R⁶ is hydrogen or C₁₋₆alkyl, preferably hydrogen; or any pharmaceutically acceptable salt thereof.

8. The method according to claim 7, wherein the compound is a diastereoisomer 2 of (2E)-4-amino-4-methylpent-2-enoic acid {(R)-1-[N-[1-(3-(N-methylcarbamoyl)-1,2,4-oxadiazol-5-yl)-2-phenylethyl]-N-methylcarbamoyl]-2-(2-naphthyl)ethyl} amide: 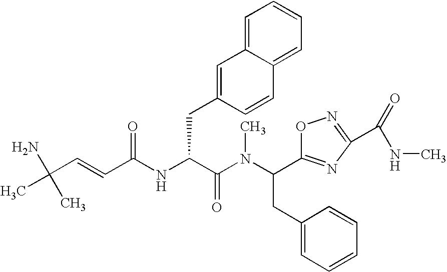
exhibiting the following ¹H-NMR spectroscopic data (DMSO-d₆, acetate salt): δ=1.30 (s, 3H); 1.32 (s, 3H); 1.95 (s, 3H); 2.55 (d, 2H); 2.80 (d, 3H); 3.00 (s, 3H); 3.30 (dd, 1H); 3.50 (dd, 1H); 5.00 (q, 1H); 6.05 (dd, 1H); 6.10 (d, 1H); 6.60 (d, 1H); 7.15-7.90 (m, 12H); 8.70 (d, 1H); and 8.95 (q, 1H); or a pharmaceutically acceptable salt thereof.

9-20. (canceled)

21. A ghrelin antagonist compound according to Formula I: 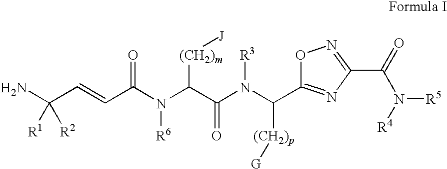
wherein R¹ and R² independently of each other are hydrogen or C₁₋₆alkyl, or R¹ and R² taken together form a C₂₋₅alkylene group; J is a group of the formula 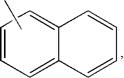
optionally substituted with one or more C₁₋₆alkyl or halogen; m is 1, 2 or 3; R³ is C₁₋₆alkyl; p is 1, 2 or 3; G is a group of the formula 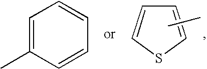
optionally substituted with one or more C₁₋₆alkyl or halogen; R⁴ and R⁵ independently of each other are hydrogen or C₁₋₆alkyl; and R⁶ is hydrogen or C₁₋₆alkyl, preferably hydrogen or a pharmaceutically acceptable salt thereof.

22. The compound according to claim 21, which is diastereoisomer 2 of (2E)-4-amino-4-methylpent-2-enoic acid {(R)-1-[N-[1-(3-(N-methylcarbamoyl)-1,2,4-oxadiazol-5-yl)-2-phenylethyl]-N-methylcarbamoyl]-2-(2-naphthyl)ethyl} amide: 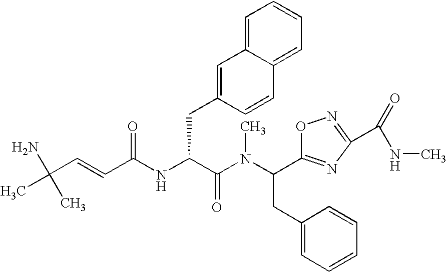
exhibiting the following ¹H-NMR spectroscopic data (DMSO-d₆, acetate salt): δ=1.30 (s, 3H); 1.32 (s, 3H); 1.95 (s, 3H); 2.55 (d, 2H); 2.80 (d, 3H); 3.00 (s, 3H); 3.30 (dd, 1H); 3.50 (dd, 1H); 5.00 (q, 1H); 6.05 (dd, 1H); 6.10 (d, 1H); 6.60 (d, 1H); 7.15-7.90 (m, 12H); 8.70 (d, 1H); and 8.95 (q, 1H); or a pharmaceutically acceptable salt thereof.

23. A pharmaceutical composition comprising, as an active ingredient, a ghrelin-antagonistic compound according to claim 21 or pharmaceutically acceptable salt thereof and a pharmaceutically acceptable carrier or diluent.

24-25. (canceled)

26. The method of claim 2, wherein the subject is a human.

27. The method of claim 7, wherein the subject is a human.


SEQUENCE LISTING (2 sequences)

SEQ ID NO: 1 - protein, 11 aa, Artificial
  Synthetic
RPKPFQWFWLL

SEQ ID NO: 2 - protein, 6 aa, Artificial
  Synthetic
HWKWFK